(19) United States
(12) Patent Application Publication
(10) Pub. No.: US 2022/0175820 A1
(43) Pub. Date: Jun. 9, 2022

(54) DEXTRAN SULFATE FOR INFLAMMATORY DERMATOSES

(71) Applicant: PIERRE FABRE DERMO-COSMETIQUE, BOULOGNE-BILLANCOURT (FR)

(72) Inventors: Marie Françoise ARIES, ESCALQUENS (FR); Stéphane POIGNY, SAUBENS (FR); Hélène HERNANDEZ-PIGEON, CUGNAUX (FR)

(73) Assignee: PIERRE FABRE DERMO-COSMETIQUE, BOULOGNE-BILLANCOURT (FR)

(21) Appl. No.: 17/600,766

(22) PCT Filed: Apr. 3, 2020
(86) PCT No.: PCT/EP2020/059527

§ 371 Date: Oct. 1, 2021

(30) Foreign Application Priority Data
Apr. 4, 2019 (FR) FR1903628

(51) Int. Cl. A61K 31/737; A61K 36/21; A61P 17/00

(52) U.S. Cl. CPC A61K 31/737; A61K 2236/19; A61P 17/00; A61K 36/21

(57) ABSTRACT

The invention relates to the use of dextran sulfate and to a dermatological composition or dermo-cosmetic composition containing dextran sulfate, in the treatment and/or prevention of inflammatory skin conditions, particularly atopic dermatitis.

### TECHNICAL FIELD

The invention concerns dextran sulfate or a dermatologically or dermo-cosmetically acceptable salt thereof, as well as a dermatological or dermo-cosmetic composition containing it, for their use in the treatment and/or prevention of inflammatory dermatoses, in particular atopic dermatitis.

### PRIOR ART

Dermatoses are skin and mucosa disorders that are characterised by unsightly manifestations such as redness and flaking patches. Several pathologies are grouped under the name of inflammatory dermatoses. Non-limiting examples include atopic dermatitis, eczema, psoriasis, rosacea, lichen planus, prurigo, seborrheic dermatitis and acne. These dermatoses very often result from inflammatory phenomena and immune disorders.

Atopic dermatitis is the skin manifestation of atopy. It is a chronic inflammatory dermatosis occurring in a genetically determined background. It affects 15 to 30% of children and 2 to 10% of adults. Its prevalence is constantly increasing in industrialized countries; it has doubled or even tripled in the past three decades and it is now considered to be a major public health concern. Atopic dermatitis is often associated with other atopic disorders, such as allergic rhinitis and asthma. This condition most often appears during early childhood and is characterized by repeated rashes over several years. It progresses by flares interspersed with spontaneous remissions. The lesions are characterized by severe skin dryness associated with inflammatory manifestations: papular, vesicular, scaly and very itchy erythematous rashes. Histologically, like many other dermatoses, atopic dermatitis is characterized by an infiltration of lymphocytes, monocytes and eosinophils around small vessels and capillaries; biochemically, it is characterized by the expression of cytokines such as thymic stromal lymphopoietin (TSLP), a major protein in triggering the inflammation associated with atopic dermatitis. Furthermore, it has been demonstrated that chemokines, especially interleukin 8 (IL8) and lipid mediators of inflammation such as prostaglandin 6kF1α (PG6KF1α), are greatly involved in dermatoses such as atopic dermatitis and in chronic inflammatory disease in general.

Eczema is an itchy dermatosis characterized by skin inflammation accompanied by redness, small blisters, flakes and itching. It may begin very early in life; it has even been observed in newborns.

Affected individuals undergo periods commonly called “eczema flares”, during which symptoms worsen. These flares, of variable duration, are interspersed with periods of remission. Eczema is a genetic disorder, but environmental factors such as the presence of chemical irritants or stress influence its onset.

Psoriasis, a classic inflammatory disease, is characterized by the appearance of thick and flaky patches of skin. These patches are present at difference areas of the body, most often on the elbows, knees and scalp. This chronic disease progresses cyclically, with periods of remission. Psoriasis can be very unpleasant and even painful when it appears on the palms or soles or in skin folds. There are several types of psoriasis, the most common form being plaque psoriasis or psoriasis vulgaris. The other forms are guttate, erythrodermic and pustular psoriasis.

Rosacea is a common chronic and progressive inflammatory dermatosis associated with vascular relaxation. It is a disorder that affects the small vessels of the face. It frequently affects people with fair skin and can have major psychological and emotional consequences. The name of this disease refers to the characteristic color of the face during the disease.

Lichen planus is an itchy rash that only affects adults. It is an inflammatory skin condition of unknown origin. This dermatosis affects the skin and mucosa as well as the hair and nails. These last two areas are generally in chronic progression which may lead to irreversible sequelae, such as alopecia and destruction of the nails.

Prurigo is intense itching of the skin with erythematous and vesicular papules with scratching lesions. This is most often an exaggerated sensitivity to insect bites, a sensitivity that lasts an abnormally long time and is especially common in young children.

Seborrheic dermatitis is a chronic inflammatory skin condition that affects areas rich in sebaceous glands, i.e., the scalp and face. It is due to a yeast, Malassezia, that is present on the skin and grows in sebum. This condition progresses in flares exacerbated by stress, lack of sun and pollution.

Acne is a common skin disease, resulting from an inflammation of pilosebaceous glands primarily due to colonization by Cutibacterium acnes in the infundibulum (Dréno et al., JEADV 2018, 32 (Suppl 2) 5-14).

In the case of mild inflammatory dermatosis conditions, emollients and keratolytics are recommended. The purpose of these treatments is to make the lesions tolerable for patients but they often only slow progression. For more severe conditions, antiinflammatories or corticosteroids, which can regulate skin inflammation, have been used for several years. All these treatments have major side effects that are sometimes very burdensome for patients. Due to the major side effects of the above-mentioned existing treatments for skin or scalp disorders resulting from a state of activation of the innate immune and inflammatory epidermal responses of the skin, there is a real need for new cosmetic active ingredients and new cosmetic compositions that can be used as a replacement for or along with treatments for said skin or scalp conditions.

### SUMMARY OF THE INVENTION

The present invention aims to respond to these needs. Indeed, completely unexpectedly, the inventors have demonstrated that dextran sulfate has pharmacological activities of interest for the treatment and prevention of inflammatory dermatoses and especially atopic dermatitis.

A first object of the invention consequently concerns dextran sulfate or a dermatologically or dermo-cosmetically acceptable salt thereof for its use in the treatment and/or prevention of inflammatory dermatoses.

Another object of the invention concerns the use of dextran sulfate or a dermatologically or dermo-cosmetically acceptable salt thereof for producing a dermatological or dermo-cosmetic composition intended for the treatment and/or prevention of inflammatory dermatoses.

Another object of the invention concerns the use of dextran sulfate or a dermatologically or dermo-cosmetically acceptable salt thereof for the treatment and/or prevention of inflammatory dermatoses.

Another object of the invention concerns a method for treating and/or preventing an inflammatory dermatosis comprising administering to a person in need thereof an effective quantity of dextran sulfate or a dermatologically or dermo-cosmetically acceptable salt thereof.

In the context of the present invention, dextran sulfate or a dermatologically or dermo-cosmetically acceptable salt thereof is advantageously obtainable or obtained by:
Fermentation of beets, especially of beet sugar, to obtain dextran, then
Sulfation of the dextran to obtain dextran sulfate, especially in the presence of magnesium sulfate, and
Optionally, salification to obtain a dermatologically or dermo-cosmetically acceptable dextran sulfate salt and, more particularly a dextran sulfate sodium salt.

The dextran sulfate or dermatologically or dermo-cosmetically-acceptable salt thereof thus obtained will advantageously have a mean molecular weight comprised between 9 kD and 20 kD.

### Definitions

Within the meaning of the present invention, “prevention” means to prevent the onset of a disease or disorder or one or more signs and/or symptoms.

The term “treatment” or “treating” of an inflammatory dermatosis means to reduce and/or inhibit the development of an inflammatory dermatosis and/or at least one of its symptoms.

In the present invention, “dermatologically or dermo-cosmetically-acceptable” means what is useful in the preparation of a dermatological or dermo-cosmetic composition, which is generally safe, nontoxic and not biologically or otherwise undesirable and which is acceptable for dermatological or dermo-cosmetic use, particularly by topical application.

In the present invention, a “dermatologically or dermo-cosmetically acceptable salt” of dextran sulfate means a dermatologically or dermo-cosmetically acceptable base addition salt formed from the sulfate functions (—OSO3H) of dextran sulfate, whose acidic proton is either replaced by a metal ion, for example an alkali metal ion (e.g. Na or K), an alkaline-earth ion (e.g. Mg or Ca) or an aluminium ion; or coordinated with a pharmaceutically-acceptable organic base such as diethanolamine, ethanolamine, N-methylglucamine, triethanolamine, tromethamine and the like; or with a pharmaceutically-acceptable inorganic base such as aluminum hydroxide, calcium hydroxide, potassium hydroxide, sodium carbonate, sodium hydroxide and the like. Advantageously it is a sodium or potassium salt, preferably a sodium salt.

“Topical application” means an application on the skin, mucosa and/or appendages.

### DESCRIPTION OF EMBODIMENTS

Dextran is a polysaccharide, and more particularly a neutral polysaccharide with no charged groups. It is a branched glucose polymer (dextrose). Advantageously, this polymer will comprise a main chain with α-1,6 glycosidic bonds between glucose monomers and branches formed by α-1,2, α-1,3 and/or α-1,4 glycosidic bonds

It can be prepared by sugar beet fermentation. It is possible to obtain it from native dextran by hydrolysis and purification of dextran fractions of different molecular weights. It can also be prepared synthetically.

Dextran can be sulfated, especially in the presence of magnesium sulfate, to give dextran sulfate.

Dextran sulfate is therefore a dextran for which at least a part of the hydroxyl groups has been replaced by sulfate groups.

Dextran sulfate or a dermatologically or dermo-cosmetically acceptable salt thereof is advantageously prepared by:
Fermentation of beets, especially of beet sugar, to obtain dextran, then
Sulfation of the dextran to obtain dextran sulfate, especially in the presence of magnesium sulfate, and
Optionally, salification to obtain a dermatologically or dermo-cosmetically acceptable dextran sulfate salt and, more particularly a dextran sulfate sodium salt.

More particularly, dextran sulfate is in the form of a sodium salt and is advantageously obtainable or obtained by:
Fermentation of beets, especially of beet sugar, to obtain dextran, then
Dissolution in acidified water, especially by formic acid, and sulfation of the dextran to obtain dextran sulfate, especially in the presence of magnesium sulfate,
Salification to obtain dextran sulfate sodium salt,
Purification, especially by solubilization in water and one or more precipitations in ethanol, and
Recovery of the dextran sulfate sodium salt, especially by centrifugation, drying (e.g., under vacuum) and grinding.

The physicochemical properties of dextran sulfate that are known in the prior art make it a good compound for cosmetic compositions, with a good solubility in water and saline solutions and high stability in solutions of pH ranging from 4 to 10 at ambient temperature. Dextran sulfate is also described as having water absorption properties, a protective effect against damage induced by free radicals, particularly by topical application, a stabilizing effect of proteins or unstable substances and a hydrating effect as a result of its excellent hydrophilic properties. Biological properties such as an anticoagulant effect, an inhibitory effect of enzymes such as hyaluronidase, glucosidases, elastase or thrombin, or an antiviral activity are also described.

Dextran sulfate can be synthetic or natural. It is understood that the dextran sulfate can be of any origin.

Preferentially, according to the invention, dextran sulfate is present in the form of a sodium salt. The INCI name is Sodium dextran sulphate and the CAS number is 9011-18-1.

According to the present invention, dextran sulfate or a dermatologically or dermo-cosmetically acceptable salt thereof advantageously has an average molecular weight comprised between 2 kDa and 5000 kDa, preferably between 4 kDa and 1000 kDa, more preferably between 5 kDa and 100 kDa, even more preferably between 9 kDa and 20 kDa, just as preferably, dextran sulfate or a dermatologically or dermo-cosmetically acceptable salt thereof has a molecular weight of between 4 kDa and 8 kDa.

Preferentially, the dextran sulfate according to the invention is provided by the SAFIC ALCAN company under the name of Dextralip 10C and is in the form of a sodium salt.

Dextran sulfate or a dermatologically or dermo-cosmetically acceptable salt thereof is useful for the treatment and/or prevention of inflammatory dermatoses.

Preferably, the inflammatory dermatoses are chosen from atopic dermatitis, eczema, psoriasis, rosacea, lichen planus, prurigo, seborrheic dermatitis and acne. Preferably it is an atopic dermatitis.

Another object of the present invention concerns a dermatological or dermo-cosmetic composition comprising as active ingredient at least one dextran sulfate or a dermatologically or dermo-cosmetically acceptable salt thereof such as defined above with at least one dermatologically or dermo-cosmetically acceptable excipient, for its use in the treatment and/or prevention of inflammatory dermatoses.

Another object of the present invention concerns the use of a dermatological or dermo-cosmetic composition comprising as active ingredient at least one dextran sulfate or a dermatologically or dermo-cosmetically acceptable salt thereof such as defined above with at least one dermatologically or dermo-cosmetically acceptable excipient, in the treatment and/or prevention of inflammatory dermatoses.

Another object of the present invention concerns the use of a dermatological or dermo-cosmetic composition comprising as active ingredient at least one dextran sulfate or a dermatologically or dermo-cosmetically acceptable salt thereof such as defined above with at least one dermatologically or dermo-cosmetically acceptable excipient, for the preparation of a medicament intended for the treatment and/or prevention of inflammatory dermatoses.

Another object of the invention concerns a method for treating and/or preventing an inflammatory dermatosis comprising administering to a person in need thereof an effective quantity of a dermatological or dermo-cosmetic composition comprising as of active ingredient at least one dextran sulfate or a dermatologically or dermo-cosmetically acceptable salt thereof such as defined above with at least one dermatologically or dermo-cosmetically acceptable excipient.

In a particular embodiment, the composition according to the invention is used in the treatment and/or prevention of an inflammatory dermatosis chosen from atopic dermatitis, eczema, psoriasis, rosacea, lichen planus, prurigo, seborrheic dermatitis and acne.

Preferably, the composition according to the invention is used in the treatment and/or prevention of atopic dermatitis.

In a particular embodiment, the dermatological or dermo-cosmetic composition according to the invention comprises 0.01 to 1%, preferentially 0.01 to 0.5%, still more preferably 0.02 to 0.3% by weight of dextran sulfate or a dermatologically or dermo-cosmetically acceptable salt thereof relative to the total weight of the composition. Advantageously, the dermatological or dermo-cosmetic composition according to the invention comprises 0.03% by dry weight of dextran sulfate or a dermatologically or dermo-cosmetically acceptable salt thereof relative to the total weight of the composition.

The compositions according to the invention are advantageously intended for topical application, especially for application on the skin.

The compositions according to the invention can thus be presented in the commonly-known forms for topical administration, i.e., notably lotions, mousses, gels, dispersions, emulsions, sprays, serums, balms, masks or creams.

Advantageously it will be a cream or balm.

The invention also relates to dermatological or dermo-cosmetic compositions according to one of the embodiments of the present invention, characterized in that they are present in an appropriate form suitable for topical application.

In addition to the sodium sulfate according to the invention, these compounds generally contain a physiologically-acceptable medium, in general based on water or solvent, for example alcohols, ethers or glycols. They can also contain surfactants, complexing agents, preservatives, stabilizers, emulsifiers, thickeners, gelling agents, humectants, emollients, trace elements, essential oils, fragrances, dyes, mattifying agents, chemical or mineral filters, moisturizers or thermal waters, etc.

The following examples illustrate the invention without limiting the scope thereof.

### EXAMPLES

### Example 1, the Effect of Dextran Sulfate in Inflammation on Prostaglandin PG6K

The keratinocyte is the most common cell in the epidermis. In response to several extracellular factors present in the environment, the epidermis releases various biologically activate mediators, in particular bioactive lipids, prostaglandins and leukotrienes that play an important role in the initiation and modulation of inflammatory skin reactions and also participate in regulating the immune response.

The keratinocyte appears to be a good model for pharmacological study of the skin. This cellular model makes it possible to determine in vitro the abilities of the various compounds to modulate the production of these mediators resulting from the metabolism of arachidonic acid.

In this study, the inventors investigated one prostaglandin in particular, 6-keto-PGF1α (PG6KF1α), which is the stable metabolite of prostacyclin. This metabolite is one of the major metabolites produced by stimulated keratinocytes and is representative of the modulation of production of arachidonic acid metabolites (Dorris and Stokes Peebles, Mediators of inflammation, vol 2012, article ID 926968, 9 pages).

The purpose of this study is to look for a potential antiinflammatory activity of dextran sulfate by measuring its effects on the release of prostaglandin PG6KF1α.

The cell line used in this study is the HaCat line (human keratinocytes).

The cells are cultured in DMEM (Dulbecco's Modified Eagle Medium) supplemented with foetal calf serum in an environment at 37° C. and 5% CO₂ on 24-well plates. They are then preincubated for 60 minutes, still at 37° C., with the products to be tested dissolved in water. A stimulant the arachidonic acid pathway is added for 5 hours. This is the stimulation phase; the stimulant is the calcium ionophore A23187 (solution in 0.01% DMSO) used at 5 μM. In this study, two different dextran sulfates are tested, a natural dextran sulfate, Dextralip® from SAFIC ALCAN with a molecular weight of 9000-20000 Da and a synthetic dextran sulfate from Welding GMBH & Co, with a molecular weight of 4000-8000 Da. After 5 hours of stimulation, still in the same medium, the culture supernatant of each well is drawn off, centrifuged at 3000 RPM then stored at −20° C. The production of prostaglandin PG6KF1α in culture supernatant is measured with the Euromedex Elisa kit according to the supplier's instructions. Statistical analysis is performed by ANOVA followed by Dunnett's post-hoc test. Note that 3 independent experiments were conducted. The results for each product and for each dose are averaged from measurements performed on 3 wells. Thus, the control group (with no stimulant) makes it possible to quantify basal PG6KF1α production. All the other groups have a stimulation period, either with no other product (making it possible to know the maximum production of PG6KF1α or in the presence of indomethacin (non-steroidal antiinflammatory) used as a reference product, or in the presence of different concentrations of dextran sulfate.

The results of PG6KF1α activation (in pg/ml) are summarized in Tables 1A and 1B below:

Table 1A: effects of a natural dextran sulfate of molecular weight 9000-20000 Da (Dextralip) on PG6KF1α production

TABLE 1A

| | Conc | PG6KF1α (pg/ml) | | | |
|---|---|---|---|---|---|
| Groups | (μg/ml) | Mean | SEM | % Inh | Stat vs stim |
| Control | — | 643.7 | 178.0 | 100 | — |
| Stimulation | — | 2560.0 | 911.7 | 0 | — |
| Indo | 0.1 μM | 839.9 | 208.4 | 33 | p < 0.01 |
| Dextran sulfate | 10 | 1878.6 | 795.3 | 36 | p < 0.01 |
| | 30 | 2113.4 | 846.5 | 23 | p < 0.01 |
| | 100 | 2287.5 | 858.6 | 14 | p < 0.05 |
| | 300 | 1950.9 | 794.9 | 32 | p < 0.01 |
| | 1000 | 1737.6 | 727.4 | 43 | p < 0.01 |
| | 3000 | 1843.8 | 733.0 | 37 | p < 0.01 |

Conc: concentration; SEM: standard error of the mean; Inh: inhibition; Indo: indomethacin.

Table 1B: effects of a synthetic dextran sulfate of molecular weight between 4000 and 8000 Da on PG6KF1α production

TABLE 1B

| | Conc | PG6KF1α (pg/ml) | | | |
|---|---|---|---|---|---|
| Groups | (μg/ml) | Mean | SEM | % Inh | stat |
| Control | — | 581.6 | 48.9 | 100 | — |
| Stimulation | — | 2053.1 | 505.8 | 0 | — |
| Dextran sulfate | 10 | 1536.5 | 472.2 | 35 | p < 0.01 |
| | 30 | 1719.7 | 472.1 | 23 | p < 0.01 |
| | 100 | 1848.5 | 528.7 | 14 | p = NS |
| | 300 | 1626.6 | 407.3 | 29 | p < 0.01 |
| | 1000 | 1462.5 | 411.5 | 40 | p < 0.01 |
| | 3000 | 1486.2 | 347.5 | 39 | p < 0.01 |

Conc: concentration;
SEM: standard error of the mean;
Inh: inhibition;
NS: not-significant

Stimulation by the calcium ionophore A23187 greatly stimulates PG6KF1α production. Indomethacin, a nonsteroidal antiinflammatory, inhibits this production by a third, which validates the relevance of this test. The two dextran sulfates tested from 10 μg/ml to 3 mg/ml show a statistically significant inhibitor effect on PG6KF1α release. This production in response to the increase of synthetic dextran sulfate concentrations resembles a bell curve. Likewise, with natural dextran sulfate, no concentration-response effect could be clearly revealed.

The inventors demonstrated the efficacy of dextran sulfate in inflammation.

### Example 2, the Effects of Dextran Sulfate in Inflammation, on NFκB Activity

The purpose of this study is to assess the potential soothing properties of dextran sulfate, at the level of the activation of transcription factor NFκB stimulated by TNFα in human keratinocytes. The cell line used in this study is the HaCat line (human keratinocytes) stably transfected with the luciferase reporter gene.

These cells are cultured on 24-well plates under the same conditions as in Example 1. They are then preincubated for 60 minutes with the products to be tested, added to the culture medium in the form of a solution in water. A stimulant, TNFα (0.3 ng/ml, diluted in culture medium) is added to the cultures which are then incubated for 5 hours at 37° C. In this study, a natural dextran sulfate, Dextralip® from SAFIC ALCAN of a molecular weight of 9000-20000 Da, is tested. A positive control is used in this test, dexamethasone (synthetic glucocorticoid hormone, with an antiinflammatory and immunosuppressant effect, added to the culture medium in the form of a solution in water) tested at 2 μM in the culture medium. Note that 3 independent experiments were conducted. The Bright-Glo™ agent (which induces cell lysis and thus enables luciferase release) and its substrate (luciferin) are added before reading the luminescence. The raw data are analyzed by Excel. The intergroup comparison is performed by one-way ANOVA followed by Dunnett's post-hoc test.

The results of NFκB activation (RLU) are summarized in Table 2 below:

TABLE 2

| | | | | RLU | | | |
|---|---|---|---|---|---|---|---|
| Groups | | conc | | Mean | sem | % Inh | Stat |
| Cont | — | — | | 1374 | 106.4 | — | — |
| Stim | — | | | 23895 | 1972.5 | — | p < 0.01 |
| Stim | Dexa | 2 | μM | 14206 | 1156.7 | 41 | p < 0.05 |
| Stim | Dex | 0.3 | mg/ml | 27024 | 1988.1 | −13 | p = NS |
| | S. | | | | | | |
| Stim | Dex | 1 | mg/ml | 23229 | 1958.5 | 3 | p = NS |
| | S. | | | | | | |
| Stim | Dex | 3 | mg/ml | 13836 | 704.7 | 42 | p < 0.01 |
| | S. | | | | | | |

RLU Relative Light Unit; Cont: control; Conc: concentration; sem: standard error of the mean; Inh: inhibition; Stim: stimulation by TNFα Dexa: dexamethasone; Dex S.: dextran sulfate; NS: not significant.

TNFα stimulation (0.3 ng/ml) does induce NFκB activation. The dexamethasone used as immunosuppressant inhibits this activation by more than 40% (p<0.05 vs without TNFα), which validates the reliability of this test.

Dextran sulfate tested at 0.3 and 1 mg/ml does not reduce NFκB activation. In return, at the concentration of 3 mg/ml, dextran sulfate significantly (p<0.01) reduces by 42% the activation of NFκB induced by TNFα.

The inventors have thus demonstrated that dextran sulfate is endowed with soothing properties.

### Example 3, Effects of Dextran Sulfate in Skin Inflammation (Atopic Dermatitis Model

Atopic dermatitis or atopic eczema is a chronic inflammatory disease progressing cyclically with phases of remission. Atopic dermatitis lesions are especially due to the activation of T cells specific to allergens. This immune response is probably due to the penetration of environmental allergens into the skin. A disruption of the skin barrier and consequently a dispersion of allergens are linked to the induction of a specific immune response and eczema lesions. Two complementary hypotheses have been proposed to explain the origin of the disease. The first hypothesis is that damage to the barrier function of the skin would allow allergens to enter and induce immune sensitization. The second hypothesis is that atopy is an immune system dysfunction leading to an imbalance of Th1/Th2 to the benefit of Th2 and a production of IgE specific to allergens. For all these reasons, atopic dermatitis is considered to be a complex disease involving several mechanisms.

In this study, the effects of a dextran sulfate according to the invention are assessed in a model mimicking an atopic dermatitis environment, on stimulated human keratinocytes.

Methods

The study is performed on normal human epidermal keratinocytes cultured under standard conditions (37° C., 5% CO₂). The culture medium is standard (keratinocyte-SFM supplemented with 0.25 ng/ml epidermal growth factor (EGF), 25 μg/ml pituitary extract and 25 μg/ml gentamycin). The test medium is the same without growth factor.

To simulate an atopic dermatitis environment, keratinocytes are preincubated for 1 hour in the medium with or without (control) the compounds to be tested, the vehicle (water) or the positive control, bafilomycin (macrolide family) at 30 nM. The dextran sulfate tested in this study is Dextralip® from SAFIC ALCAN of a molecular weight of 9000-20000 Da. After this preincubation, keratinocytes are stimulated for 24 hours by a mixture of TLR ligands (Poly I:C and PamC3) and inflammatory cytokines (IL-4 and IL-13) added to the cells. An unstimulated control condition is also done at the same time. The cells are incubated for 24 hours.

The culture supernatants are collected, centrifuged and frozen at −80° C. TSLP and IL-8 are quantified by ELISA.

Three independent experiments are conducted. Statistical analysis is determined by one-way ANOVA followed by Dunnett's post-hoc test.

Results

The production of TSLP by normal human epidermal keratinocytes measured at 24 hours is shown in Table 3 below.

TABLE 3

| TSLP production at 24 h | | | Mean (pg/ml) | sem | % Inh |
|---|---|---|---|---|---|
| Control | | | 9 | 3 | — |
| Stimulation | | | 248 | 70 | — |
| Bafilomycin | 30 | nM | 81 | 30 | 70* |
| Dextran sulfate | 0.3 | μg/ml | 247 | 84 | 0 |
| | 3 | μg/ml | 34 | 6 | 90** |
| | 30 | μg/ml | 19 | 1 | 96** |

sem: standard error of the mean;
Inh: inhibition;
*p < 0.05;
**p < 0.01 vs simulation.

Keratinocyte stimulation by a cocktail of agents (Poly I:C, PamC3, IL-4 and IL-13) to mimic an atopic dermatitis environment does induce increased TSLP production at 24 hours. Bafilomycin, used as positive control, significantly inhibits TSLP production. These results fully validate this pharmacological test.

Dextran sulfate according to the invention almost completely inhibits TSLP production measured at 24 hours. Indeed, at 3 μg/ml dextran sulfate reduces TSLP production by 90% and up to 96% at 30 μg/ml (p<0.01 for the two concentrations, versus the stimulated condition in Table 3). In return, the ten-fold lower concentration of dextran sulfate does not induce reduction in TSLP production. The inhibitor effect of dextran sulfate on TSLP production induced by an atopic dermatitis environment appears to be clearly concentration dependent.

The production by normal human epidermal keratinocytes of interleukin 8 (IL-8) measured at 24 hours is shown in Table 4 below.

TABLE 4

| Production of IL-8 at 24 h | | | Mean (pg/ml) | sem | % Inh |
|---|---|---|---|---|---|
| Control | | | 0 | 0 | — |
| Stimulation | | | 59647 | 6889 | — |
| Bafilomycin | 30 | nM | 26022 | 4932 | 56** |
| Dextran sulfate | 0.3 | μg/ml | 63672 | 11903 | 0 |
| | 3 | μg/ml | 10837 | 2797 | 82** |
| | 30 | μg/ml | 170 | 170 | 100** |

sem: standard error of the mean;
Inh: inhibition;
**p < 0.01 vs simulation.

The stimulation of keratinocytes by the cocktail of agents to mimic an atopic dermatitis environment induces a massive production of IL-8. Bafilomycin (30 nM) significantly inhibits the production of IL-8 (more than 50% reduction). All of these results validate the pharmacological test.

Dextran sulfate according to the invention reduces IL-8 production induced by an atopic dermatitis environment in a concentration-dependent manner. The lowest dextran sulfate concentration tested is not sufficient to inhibit IL-8 production (Table 2). In return, at 3 μg/ml, dextran sulfate significantly reduces IL-8 production by more than 80% (p<0.01 versus the stimulation condition). At 30 μg/ml dextran sulfate completely inhibits IL-8 production (100% inhibition), showing that dextran sulfate according to the invention is very effective for reducing these selective cytokines of atopic dermatitis.

Conclusion of these Studies

The inventors have thus shown that normal human epidermal keratinocytes produce a significant quantity of TSLP and IL-8 after 24 hours of stimulation by a cocktail of agents (Poly I:C, PamC3, IL-4 and IL-13) mimicking an atopic dermatitis environment. In this model, the inventors demonstrate that dextran sulfate according to the invention is very effective and is able to prevent this release of inflammatory cytokines and especially TSLP, key marker in the development of atopic dermatitis, demonstrating a protective role in this disease.

### Example 4, Assessment of Dextran Sulfate in the Modulation of the Genes Involved in Keratinocyte Differentiation and Hydration

The epidermis plays a major protective role as both a mechanical and chemical barrier for the body. It ensures that an impermeable skin barrier function is maintained. Corneocytes, the keratinocytes of the stratum corneum, together with a lipid matrix, ensure this function for the most part. Nevertheless, deeper layers also contribute to building the elements inherent in this function. The differentiation ability of epidermal keratinocytes ensures the construction of a barrier having the function of selective permeability (Elias and Choi, Exp. Dermatol. 14(10), p 719-726, 2005).

The differentiation of keratinocytes is regulated in space and time, from the deepest layers of the skin, the basement membrane being the least differentiated, to the stratum corneum, the final step of keratinocyte differentiation into corneocytes (Houben et al., Skin Pharmacol. Physiol. 20(3), p 122-132, 2007). From the cellular and molecular viewpoint, the formation of keratin filaments, the transformation of keratinocytes into corneocytes, or “keratinization”, and the formation of an intercellular lipidic cement of lamellar structure are primarily observed, ensuring the impermeability and function of the skin barrier.

In terms of proteins, epidermal differentiation is mainly concentrated on the development of structural cytoplasm proteins such as cytokeratins, which contribute to the architectural integrity of the epidermis. Their expression varies according to the degree of maturation of the keratinocytes. Basic keratin 1 and acidic keratin 10 are early markers of terminal keratinocyte differentiation, present in the suprabasal layers of the epidermis. The expression of other markers in this biological process, which takes place later, can be followed in the same way as for the horny envelope, such as involucrin, together with certain principal enzymes causing structural proteins to form bridges among themselves and with keratinocyte lipids and transglutaminases (TGM), such as TGM1 or 3 (Houben et al., Skin Pharmacol. Physiol. 20(3), p 122-132, 2007).

The fibrous matrix present in the corneocytes is formed during the transition between keratinocytes of the stratum granulosum and corneocytes. Loricrin is a structural protein containing glutamine and lysine residues that permit adherence to other proteins in the horny envelope. Basic filaggrin molecules produced from their precursor, profilaggrin, stored in keratohyalin granules, combine with cytokeratin filaments, to then be able to aggregate. Filaggrin, degraded by caspase 14, also represents the primary source of several major constituents of the natural hydration factor in the stratum corneum. Other markers are specific for differentiated keratinocytes. Claudin 4 (CLDN4) is one of the tight junction proteins. It is essentially expressed in the stratum granulosum and its expression is increased during keratinocyte differentiation. Corneodesmosin (CDSN) is expressed in corneocytes. It is an essential protein of corneodesmosomes and its proteolysis is necessary for desquamation. Kallikreins, such as KLKS and KLK7, exhibit activity similar to that of chymotrypsin and play a role in the proteolysis of intercellular cohesion structures that precede desquamation, i.e., the elimination of the outermost layer of the stratum corneum.

At the same time, the synthesis and transport of keratinocyte lipids form the base of the intercorneocyte lipid cement essential to the skin barrier, whose formation is the final phase in terminal epidermal differentiation. This extracellular lipid matrix is the main barrier for the transcutaneous transport of fluids and electrolytes (Feingold, J. Lipid Res. 48, p 2531-2549, 2007). Thus, a certain number of enzymes and lipid transporters have their keratinocyte expression upregulated together with differentiation. The cement results from the equilibrium between three lipid species, i.e., cholesterol, free fatty acids and ceramides. These lipids originate from glucosylceramides, sphingomyelin, cholesterol and phospholipids produced in the stratum spinosum and the stratum granulosum. They are transported by the lamellar bodies, which are secretory organelles and which fuse with the stratum granulosum and the stratum corneum. In addition to these lipid precursors, the lamellar bodies contain numerous enzymes, including lipidases such as a acidic sphingomyelinase, beta-glucocerebrosidase or phospholipidases A2, together with acidic and neutral lipases. Delivered at the same time as the lipid precursors into the extracellular spaces, these enzymes respectively convert sphingomyelin into ceramide, glucocerebrosides into ceramides and phospholipids into free fatty acids and glycerol. SULT2B1 is a sulfotransferase cholesterol expressed in differentiated keratinocytes and is involved in the synthesis of cholesterol sulfate. A recent study has also revealed that cholesterol sulfate induces the expression of filaggrin by an increased expression of RORα (Hanyu et al., Biochem. Biophys. Res. Commun. 428(1), p 99-104, 2012).

Epidermal ceramides play a specific and major role and represent an essential marker of the functionality of the skin barrier. The enzymes playing a role in ceramide production of the skin have their expression and activity increased specifically when the barrier function is altered and together with the degree of epidermal differentiation (Feingold, 2007). This is the specific case of aSmase and β-glucoceramidase, involved in the extracellular metabolism of skin ceramides. UGCG (UDP-glucose ceramide glucosyltransferase) is also involved in the synthesis of glucosylceramides. UGCG catalyzes the first glycosylation step in the biosynthesis of glycosphingolipids and is necessary for the regular arrangement of lipids and proteins in the lamellar bodies and for the maintenance of the epidermal barrier (Jennemann et al. J. Biol. Chem. 282(5), p 3083-3094, 2007). DGS2 (sphingolipid C4-hydroxylase/delta-4 desaturase) acts as both sphingolipid C4-hydroxylase and as delta-4 desaturase; its dihydroceramide hydroxylase activity competes with the production of the phytoceramides of human skin.

FABP-E (FABP5), protein for binding to epidermal fatty acids, is a lipid transporter. FABP-E plays an important role in keratinocyte differentiation.

One of the functions of water in the stratum corneum is to active enzymatic hydrolysis reactions necessary to skin suppleness and normal desquamation (Rawlings and Matts J. Invest. Dermatol. 124(6), p 1099-1110, 2005). If the water content in the stratum corneum falls below a critical level, enzymatic reactions are disrupted, leading to an adherence of corneocytes and accumulation of cells on the skin surface. This creates visible dryness and itching and the skin peels and exfoliates.

The barrier function of the skin also includes defence against microorganisms. The epithelium plays an active role in the host's innate defences. Cutaneous antimicrobial systems rely, among other things, on the presence of certain surface lipids and certain constituent proteins which are increasingly expressed according to the state of differentiation of keratinocytes, such as RNAse 7 or proteinase inhibitor 3. These proteins have antimicrobial activities. There is also an adaptive component of innate immunity relying on the inducible secretion of antimicrobial peptides that have direct antimicrobial activities. They play an important role as inflammation mediators by having effects on epithelial and inflammatory cells. Antimicrobial peptides are generally synthesised in the upper layers of the stratum spinosum and the stratum granulosum but they are active in the stratum corneum where they are released. The most studied antimicrobial peptides of the skin are β-defensins and cathelicidins. Human β-defensins are the major class of antimicrobial peptides found in human epitheliums and four of them have been identified in the skin, hBD 1-4. Although they belong to the same family, they are regulated by different pathways. Human β-defensin 2 (hBD-2 or DEFB4A), a 4 kDa peptide binding heparin, is one of the main cutaneous antimicrobial peptides.

Skin hydration relies on two points, transdermal water supply from the skin bloodstream and epidermal water retention, which involves the barrier function. However the barrier to water loss is not infallible. A normal exchange of water between the external and internal environments through the stratum corneum is called transepidermal water loss (TEWL) and is inherent to insensible water loss (IWL).

Normal human keratinocytes were incubated for 48 h with dextran sulfate dissolved in culture medium at 2 mg/ml. The dextran sulfate tested in this study comes from Welding GMBH & Co; its molecular weight is 4000-8000 Da. The effects of this compound were assessed via the RT-qPCR technique with the analysis of 12 target genes chosen for their importance in keratinocyte differentiation, antimicrobial defence and hydration. Calcium chloride (tested at 1.5 mM) is used as the reference compound (physiological agent inducing terminal epidermal differentiation). The cells are then collected for analysis of target expression (mRNA) by real-time PCR. Total RNAs were extracted with TriPure Isolation Reagent® according to the supplier's instructions (Roche Life Science, Meylan, France). They were assayed with the Bioanalyser 2100 (Agilent Technologies, Les Ulis, France). The mRNAs were reverse transcribed into complementary DNA with oligo(dT) and Transcriptor Reverse Transcriptase. PCR was performed in the LightCycler® System according to the supplier's instructions (Roche). The PCR mix used is SYBR green I. The results were analyzed with Microsoft Excel®. The relative quantity is calculated for each gene by normalizing with two reference genes, ribosomal protein L13A (RPL13A) and TATA box binding protein (TBP).

Results

Since some markers are expressed very weakly in the control cells, the relative quantity could be very high and variable according to the experiment.

The inventors showed that after 48 h of incubation, dextran sulfate tested at 2 mg/ml significantly induces different markers of lipid differentiation of keratinocytes (induction of SULT2B1, FABP5, DGS2), numerous markers of protein differentiation of keratinocytes (induction of KLK7, FLG, TGM1, CASP14 and CLDN4), induces antimicrobial peptide hBD2 (or DEFB4) and finally induction of hydration markers (FLG and CASP14). All these results are shown in Table 5 below.

TABLE 5

| | |
|---|---|
| Calcium chloride | Dextran sulfate |
| (1.5 mM) | (2 mg/ml) |

| Genes | Mean RQ | sem | Mean RQ | sem |
|---|---|---|---|---|
| SULT2B1 | 14.0 | 0.7 | 41.4 | 37.0 |
| FABP5 | 6.9 | 3.9 | 25.4 | 6.7 |
| DEGS2 | 3.0 | 1.3 | 63.8 | 42.3 |
| KLK7 | 11.3 | 2.8 | 96.0 | 20.1 |
| FLG | 4.5 | 3.4 | 81.2 | 58.6 |
| CDSN | 1.8 | 0.5 | 8.0 | 2.0 |
| TGM1 | 9.0 | 0.9 | 27.0 | 17.0 |
| CASP14 | 1.6 | 0.8 | 23.8 | 16.8 |
| CLDN4 | 2.5 | 0.4 | 9.2 | 3.8 |
| DEFB4A | 118.0 | 109.8 | 41.8 | NV |

RQ: relative quantity; sem: standard error of the mean; NV: not validated; SULT2B1: cholesterol sulfotransferase; FABP5: epidermal fatty acid binding protein; DEGS2: sphingolipid C4-hydroxylase/delta-4 desaturase; KLK7: type 7 kallikrein ; FLG: filaggrin; CDSN : corneodesmosin; TGM1: transglutaminase 1; CASP14: caspase 14; CLDN4: claudin 4; DEFB4A: β-defensin 2.

The inventors thus show that dextran sulfate acts effectively by activating keratinocyte differentiation and by inducing the expression of antimicrobial peptides. This dual effect makes it possible to conclude that dextran sulfate considerably restores the skin barrier, strengthens the skin's microbial defences and prevents skin dehydration. The inventors demonstrate that dextran sulfate is useful in the treatment and/or prevention of an inflammatory dermatosis.

1-14. (canceled)

15. A method for treating and/or preventing an inflammatory dermatosis comprising administering to a person in need thereof an effective quantity of dextran sulfate or a dermatologically or dermo-cosmetically acceptable salt thereof obtainable by:
fermenting beets to obtain dextran, then
sulfating the dextran to obtain dextran sulfate, and
optionally, salifying the dextran sulfate to obtain a dermatologically or dermo-cosmetically acceptable dextran sulfate salt.

16. The method according to claim 15, wherein sulfating the dextran is performed in the presence of magnesium sulfate.

17. The method according to claim 15, wherein the dextran sulfate has a molecular weight of 2 kDa to 5000 kDa.

18. The method according to claim 15, wherein the dextran sulfate has a molecular weight of 9 kDa to 20 kDa.

19. The method according to claim 15, wherein the dextran sulfate is in the sodium salt form.

20. The method according to claim 15, wherein the inflammatory dermatosis is chosen from atopic dermatitis, eczema, psoriasis, rosacea, lichen planus, prurigo, seborrheic dermatitis and acne.

21. The method according to claim 15, wherein the inflammatory dermatosis is atopic dermatitis.

22. A method for treating and/or preventing an inflammatory dermatosis comprising administering to a person in need thereof an effective quantity of a dermatological or dermo-cosmetic composition comprising as active ingredient at least one dextran sulfate or a dermatologically or dermo-cosmetically acceptable salt thereof, with at least one dermatologically or dermo-cosmetically acceptable excipient,
wherein the dextran sulfate or the dermatologically or dermo-cosmetically acceptable salt thereof is obtainable by:
fermenting beets to obtain dextran, then
sulfating the dextran to obtain dextran sulfate, and
optionally, salifying the dextran sulfate to obtain a dermatologically or dermo-cosmetically acceptable dextran sulfate salt.

23. The method according to claim 22, wherein sulfating the dextran is performed in the presence of magnesium sulfate.

24. The method according to claim 22, wherein the dextran sulfate or the dermatologically or dermo-cosmetically-acceptable salt thereof has a molecular weight of 2 kDa to 5000 kDa.

25. The method according to claim 22, wherein the dextran sulfate or the dermatologically or dermo-cosmetically-acceptable salt thereof has a molecular weight of 9 kDa to 20 kDa.

26. The method according to claim 22, wherein the dermatological or dermo-cosmetic composition contains 0.01 to 0.5% by weight of the dextran sulfate or the dermatologically or dermo-cosmetically acceptable salt thereof relative to the total weight of the composition.

27. The method according to claim 22, wherein the dextran sulfate is in the sodium salt form.

28. The method according to claim 22, wherein the inflammatory dermatosis is chosen from atopic dermatitis, eczema, psoriasis, rosacea, lichen planus, prurigo, seborrheic dermatitis and acne.

29. The method according to claim 22, wherein the inflammatory dermatosis is atopic dermatitis.

30. The method according to claim 22, wherein the dermatological or dermo-cosmetic composition is in an appropriate form for topical administration.
